(19) United States
(12) Patent Application Publication
(10) Pub. No.: US 2007/0141550 A1
(43) Pub. Date: Jun. 21, 2007

(54) Immobilized-enzyme microreactor devices for characterization of biomolecular analytes and associated methods

(76) Inventors: Richard N. Zare, Stanford, CA (US); Maria T. Dulay, Sunnyvale, CA (US); Quentin J. Baca, Albuquerque, NM (US); Gabriele S. Trapp, Coeppingen (DE)

Correspondence Address: PARSONS HSUE & DE RUNTZ LLP, 595 MARKET STREET, SUITE 1900, SAN FRANCISCO, CA, 94105, US

(21) Appl. No.: 10/582,646

(22) PCT Filed: Dec. 13, 2004
(86) PCT No.: PCT/US04/41640

§ 371 Date: Dec. 20, 2006

(30) Foreign Application Priority Data
Dec. 12, 2003 (US) 10/734998

(51) Int. Cl. C12Q 1/68; G01N 33/53; C12Q 1/00; C12M 3/00; B05D 3/02

(52) U.S. Cl. 435/4; 435/7.1; 435/287.2; 427/2.11; 977/924; 435/6

(57) ABSTRACT

A method that comprises providing a polymerized sol-gel material (PSG) and linking an enzyme to a surface of the PSG via covalent linkage is provided. The surface of the PSG is derivatized with a linker that comprises a functional group for linking itself to the surface of the PSG and a functional group for linking itself with the enzyme. The linked-enzyme PSG, or microreactor, is an effective means of at least partially digesting a substrate, such as a biological substrate. The activity of the enzyme of the microreactor may be significantly enhanced, up to 200-fold for example, relative to the activity of the enzyme free of the microreactor. The microreactor is thus an effective vehicle for digesting a substrate such as a biomolecule, a protein, an oligonucleotide, a peptide, a steroid, and/or an organic acid, after which, any remaining substrate and one or more digestion product(s) may be separated and detected. Microreactors and integrated devices that incorporate microreactors, such as columns, pipet tips, wells, and well-plates, are also provided.

### CROSS-REFERENCE TO RELATED APPLICATIONS

This application is related to U.S. application Ser. No. 10/124,654, filed on Apr. 16, 2002, which is a continuation-in-part of U.S. application Ser. No. 09/929,275, filed on Aug. 13, 2001, and of U.S. application Ser. No. 10/008,482, filed on Nov. 13, 2001, the latter of which is a continuation of U.S. application Ser. No. 09/507,707, filed on Feb. 18, 2000. This application is also related to U.S. application Ser. No. 10/674,652, filed on Sep. 29, 2003, which is a continuation of the aforementioned U.S. application Ser. No. 10/008,482, which is a continuation of aforementioned U.S. application Ser. No. 09/507,707. This application is further related to U.S. application Ser. No. 09/978,515, filed on Oct. 15, 2001, which is a continuation-in-part of the aforementioned U.S. application Ser. No. 09/929,275. Each of the foregoing applications is incorporated herein in its entirety by this reference.

### FIELD OF THE INVENTION

The present invention generally relates to the preparation of a sol-gel material, such as a sol-gel material suitable for use in a sieve or separation channel that is used in separation processes, such as chromatographic or electrophoretic separation processes. The invention further relates to a sieving or separation channel, a method of preparing a sieving or separation channel, and a method of sieving or separating at least one analyte from a medium. The invention particular relates to the preparation of a sol-gel material suitable for the separation of a sample, such as a biomolecular sample, comprising at least one proteinaceous or peptidic analyte, and associated devices and methods.

### BACKGROUND OF THE INVENTION

Chromatographic materials are used in a variety of separation processes, such as high-performance liquid chromatography (HPLC) and capillary electrochromatography (CEC), which are used to separate analytes in a medium. By way of example, modified silica particles are typically used as the active chromatographic materials in HPLC. In recent years, CEC has been shown to be a complementary and orthogonal technique to HPLC, often providing better and more efficient separation of analytes.

Generally, two classes of chromatographic stationary phases have been used in HPLC and CEC, namely: (1) stationary phases comprised of silica particles, such as those typically used in HPLC; and (2) stationary phases comprised of monoliths, such as those prepared via photochemical and/or sol-gel techniques. Monolithic stationary phases have received the most attention because they are easy to prepare in small diameter fused-silica capillaries that are used for CEC. By way of example, a monolithic stationary phase can be prepared in a capillary by providing a solution comprising a mixture of one or more monomer(s), one or more porogenic solvent(s), and one or more photoinitiator(s) in a capillary, and irradiating the solution with light to photopolymerize it, thereby producing a photopolymerized monolith. The porous characteristics of a monolith, such as the size of the pores in the polymer matrix of the monolith, may be controlled by the amount of cross-linking monomer in the monolith, the quality of the porogenic solvent, and the molar ratio of the monomer and the porogenic solvent. (See C. Viklund et al., “Molded” Macroporous Poly(glycidyl methacrylate-co-trimethylol-propane trimethacrylate) Materials with Fine Controlled Porous Properties: Preparation of Monoliths Using Photoinitiated Polymerization, Chem. Mater. 1997, 9, (2) 463-471, hereinafter referred to as “C. Viklund et al.”)

A monolithic stationary phase can be purely inorganic. (See F. Svec et al., Monolithic Stationary Phases for Capillary Electrochromatography Based on Synthetic Polymers: Designs and Applications, J. High Resol. Chromatogr. 2000, 23, (1) 3-18 and the references listed therein.) For example, an inorganic monolith has been prepared by providing a solution comprising methacrylate-substituted monomers, and irradiating the solution with ultraviolet light to form a photopolymerized inorganic monolith. (See C. Viklund et al.)

Alternatively, a monolithic stationary phase can be a mix, or a hybrid, of organic and inorganic components. (See M. T. Dulay et al., Photopolymerized Sol-Gel Monoliths for Capillary Electrochromatography, Anal. Chem. 2001, 73, (16) 3921-3926 and the references listed therein, hereinafter referred to as “M. T. Dulay et al.”) Hybrid organic-inorganic monoliths have been prepared using a metalorganic compound, such as a metal alkoxide, in a sol-gel process. (See C. J. Brinker et al., Sol-Gel Science, Academic Press, Inc. (1990), hereinafter referred to as “C. J. Brinker et al.”) This process may involve a hydrolysis reaction, in which a metal alkoxide source is reacted with water and/or a non-aqueous solvent to produce partially hydrolyzed molecules, and a condensation reaction, in which the partially hydrolyzed molecules become linked to form a polymer and polymerization proceeds until a gel is formed. (C. J. Brinker et al.) In this process, the metal alkoxide source is an alkoxy compound represented by the formula, RₙM(OR′)₄₋ₙ, where R is a proton or other ligand, M is a metal or metalloid atom, O is an oxygen atom, R′ is an alkyl, such that OR is an alkoxy group, n is a number from zero to less than 4, and R and R′ may be the same or different. (C. J. Brinker et al.) A hybrid organic-inorganic monolith has also been prepared by providing a solution comprising a methacrylate-substituted, metal alkoxide monomer, and irradiating the solution with ultraviolet light to form a porous organic-inorganic photopolymer. (M. T. Dulay et al.)

The above-described monoliths are useful for the separation of a variety of analytes. However, when the separation concerns a biological sample containing a protein analyte or several protein analytes, additional considerations generally come into play. For example, complete structural characterization of the protein, which is a complex compound composed of amino acids, typically involves a combination of chemical, analytical and instrumental techniques. On the chemical side, the protein is typically broken down into smaller peptide fragments, such as by enzymatic digestion of the protein, and the peptide fragments are then analyzed by a variety of methods. The random immobilization of enzymes on solid surfaces of monolithic substrates to facilitate such enzymatic digestion has been investigated, however, the immobilization procedure has been shown to decrease the activity of the enzyme. (See M. Vodopivec et al., Characterization of CIM monoliths as enzyme reactors, J. Chrom. B 2003, 795, 105-113, and the references listed therein.) On the analytical and instrumentational side, the amount of protein available in a biological sample is often minute, which places a limitation on current analysis techniques and instrumentation. Attempts to address this limitation include using analytical techniques, such as capillary electrophoresis (CE), and instrumentation, such as HPLC and mass spectrometry, of increased sensitivity.

Further development of sol-gel materials, and devices and methods associated therewith, particularly those associated with protein and peptide characterization, is desirable.

### SUMMARY OF THE INVENTION

This invention relates to a method of preparing an analytical device for the characterization of a biomolecule and its components, such as a protein and/or a peptide. The device, or microreactor, comprises a solid support to which an enzyme is linked, or on which an enzyme is immobilized. According to one aspect of the invention, the microreactor generally comprises a polymerized or a photopolymerized sol-gel solid support having a surface to which an enzyme is covalently bound. The microreactor may be prepared by forming a photopolymerized sol-gel monolith of a specific length, derivatizing the monolith surface using a silicate reagent, and reacting an enzyme with a reactive functional group associated with the surface-bound silicate reagent.

As the preparation of the linked-enzyme microreactor need not involve heating, or any significant heating, the enzyme is not damaged, or diminished in its enzymatic activity. In fact, it has been demonstrated that the activity of the enzyme in a linked-enzyme microreactor according to the present invention may be increased significantly.

According to various aspects of the invention, the microreactor may be integrated into another device, such as a separation channel or a micropipet tip, for example. The immobilized-enzyme device, or integrated devices comprising the immobilized-enzyme device, has been shown to increase the rate of enzymatic digestion of a protein substrate and/or a peptide substrate, and thus, improve protein and/or peptide characterization.

These and various other aspects, features and embodiments of the present invention are further described herein.

### BRIEF DESCRIPTION OF THE DRAWINGS

A detailed description of various aspects, features and embodiments of the present invention is provided herein with reference to the accompanying drawings, which are briefly described below. The drawings are illustrative and are not necessarily drawn to scale. The drawings illustrate various aspects or features of the present invention and may illustrate one or more embodiment(s) or example(s) of the present invention in whole or in part. A reference numeral, letter, and/or symbol that is used in one drawing to refer to a particular element or feature may be used in another drawing to refer to a like element or feature.

FIG. 1 is a schematic representation of a synthesis of a photopolymerized sol-sel material, or monolith.

FIG. 2 is an illustration of a column that incorporates a microreactor, according to an embodiment of the present invention.

FIG. 3 is a photograph of a tip that incorporates a microreactor, according to an embodiment of the present invention.

FIG. 4 is a schematic representation of a preparation of a microreactor, comprised of a derivatized, photopolymerized sol-gel material, or monolith, having an enzyme attached thereto, according to an embodiment of the present invention.

FIG. 5A, FIG. 5B, FIG. 5C and FIG. 5D, which may sometimes be collectively referred to as FIG. 5 herein, are schematic illustrations of a use of a column that incorporates a microreactor, according to an embodiment of the invention.

FIG. 6 is a schematic representation of an enzymatic digestion of a sample using a microreactor, according to an embodiment of the present invention, as further described herein in relation to an Experimental Example.

FIG. 7A and FIG. 7B are graphical representations of results obtained in connection with the enzymatic digestion schematically represented in FIG. 6, as further described herein in relation to an Experimental Example.

FIG. 8 is a graphical representation of results obtained in connection with another enzymatic digestion of a sample using a microreactor, according to an embodiment of the present invention, as further described herein in relation to an Experimental Example.

FIG. 9 is a graphical representation of results obtained in connection with an enzymatic digestion of a sample using a microreactor, according to an embodiment of the present invention, as further described herein in relation to an Experimental Example.

FIG. 10 is a schematic illustration of the incorporation of a microreactor in a well of a well plate, according to an embodiment of the present invention.

### DETAILED DESCRIPTION OF THE INVENTION

The present invention provides an immobilized-enzyme device, or microreactor, for characterization of a biomolecule or a component thereof, such as a protein and/or a peptide. The invention further provides a method of preparing such a device and a method of using such a device.

In the description of the invention herein, it will be understood that a word appearing in the singular encompasses its plural counterpart, and a word appearing in the plural encompasses its singular counterpart, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives, is merely illustrative, not limiting, unless implicitly or explicitly understood or stated otherwise.

According to one aspect of the invention, an enzyme is immobilized on the surface of a polymerized sol-gel (PSG) material, and preferably, a photopolymerized sol-gel material. Such polymerized sol-gel materials are porous and are amenable to design or modification to achieve desirable porous characteristics. A photopolymerized sol-gel material is preferred, as it can be prepared and modified at low temperature, such that problems associated with higher temperatures, such as the cracking of the material or the thermal degrading, deactivation, and/or destruction of modifiers, are reduced, minimized, or avoided.

The enzyme may be immobilized relative to the PSG material by way of covalent bonds or linkages formed between the enzyme and the surface of the PSG material. Covalent bonds are used, as non-covalent bonds or linkages, such as those associated with ionic or electrostatic interactions, or with van der Waals forces, are more likely to push the enzyme out somewhat from the PSG material and are much less permanent. Further, covalent chemical bonds are used in preference to physical enzyme-immobilizing or capturing vehicles, such as enzyme-entrapment or encapsulation. (See for example, D. Avnir et al., Enzymes and Other Proteins Entrapped in Sol-Gel Materials, Chem. Mater. 1994 (Invited Review), 6, 1605-1614.) This is because, comparatively, the covalent chemical bonds provide more robust and stable enzyme immobilization. Additionally, it is believed that chemical immobilization of enzymes may reduce or minimize enzyme autolysis, as the enzymes, by virtue of this chemical immobilization, are spatially distant enough to reduce, minimize, or avoid enzyme self-degradation. Thus, as used herein, any reference to linkage, bonding or immobilization of the enzyme relative to the surface of the PSG material, or any associated or corresponding terms, generally refers to covalent linkage, bonding or immobilization, via chemical means.

A greater amount of enzyme covalently bound to the surface of the PSG material generally corresponds to a greater amount of substrate, whether protein, peptide, or artificial substrate, that is hydrolyzed. Even so, even a small amount, such as an amount on the order of less than micromolar, such as nanomolar or picomolar, for example, of enzyme bound to the PSG material in this manner, will result in a higher rate of hydrolysis, as compared to the same amount of enzyme in bulk solution.

In one example, oligomers of methacrylate-substituted silicate reagents are formed using sol-gel chemistry. By way of example, such an oligomer may be an oligomer of tetramethylorthosilicate. The oligomers are photopolymerized to provide a PSG material comprising a porous network of both organic and inorganic components. Alternatively, an inorganic PSG material, such as composed only of tetralkoxysilane, for example, may be used, although in such a case, the PSG monolith itself does not play an active role in separation. Such inorganic PSG material would not be used for CEC, for example. The PSG material is then derivatized with a linker, as further described herein, following which an enzyme is immobilized on the PSG material to provide an immobilized-enzyme microreactor. The photopolymerization process determines the dimensions, such as a length, of the immobilized-enzyme microreactor, as further described herein.

According to one aspect of the present invention, an appropriate PSG material is derivatized with a linker that comprises a reactive functional group. The derivatization process generally involves the functionalization of the PSG monolith, via its free silanol groups, with a suitable linker that has a reactive functional group suitable for interaction with a suitable enzyme. An example of a suitable linker is an alkoxysilane, such as a silicate, that comprises an aldehyde or a succinimide group. An enzyme having an active amine functional group is then reacted with the derivatized PSG material. Examples of suitable enzymes having an active amine functional group include trypsin, pepsin, and chymotrypsin. Any unreacted linker is rinsed from the derivatized PSG material using a suitable rinsing agent, such as toluene or another appropriate organic solvent, for example.

When the linker comprises an aldehyde functional group, the amine of the enzyme and the aldehyde on the PSG surface react at low temperature, such as from about 4° C. to about 23° C., or room temperature (about 20° C. to about 25° C.), to form an imine bond, typically in a period of from about 30 to about 60 minutes. This reaction is associated with Schiff chemistry. When the linker comprises a succinimide functional group, the amine of the enzyme and the succinimide on the PSG surface readily react at this low temperature. By way of example, the reaction may take place in a relatively short period of from about 30 to about 60 minutes at a temperature of from about 4° C. to about 23° C., or room temperature (from about 20° C. to about 25° C.). Use of a linker with a succinimide functional group may be advantageous in that the resulting PSG-bound enzyme is less susceptible to water hydrolysis than is a PSG-bound enzyme resulting from Schiff chemistry. Regardless of the type of derivatization reaction employed, the reaction between an enzyme and the derivatized PSG material results in the binding of the enzyme to the PSG surface, preferably via enzyme-immobilizing covalent bounds.

According to another aspect of the invention, a device comprising an immobilized-enzyme microreactor that is integrated in a separation channel is provided. The separation channel can be a fused-silica capillary column that is useful for CE separations. Merely by way of example, a 75-μm inner diameter×365-μm outer diameter fused-silica capillary from Polymicro Technologies of Phoenix, Ariz. may be used. The microreactor can be of any length, such as from about 1 cm to about 10 cm in length, merely by way of example. Once the microreactor is integrated into the separation channel, a sample containing a peptide substrate or a protein substrate that is suitable for digestion by the immobilized enzyme is introduced into the microreactor. This introduction may be facilitated by the application of any suitable pressure or voltage. Merely by way of example, a suitable pressure may be from about 0.5 psi to about 20 psi, which may depend on the instrumentation employed, and a suitable voltage may be from about 1 kV to about 5 kV.

The digestion reaction can be completed within seconds or minutes, such as from about 5 seconds and about 60 minutes, such as from about 5 seconds to about 15 minutes, depending on the reaction temperature, which may be from about 15° C. to about 40° C., for example. The digestion reaction may take longer, although this is not generally desired, as one of the advantages of the invention is the speed of the digestion reaction. A higher reaction temperature generally corresponds to a faster reaction. Longer reaction times, such as those from about 15 minutes to about 60 minutes or even longer, merely by way of example, may be used where it is desired to preconcentrate the sample.

Once the digestion reaction is completed, a voltage is applied across the capillary column, such that the digestion product(s) and any remaining substrate are separated by capillary electrophoresis. Merely by way of example, a suitable voltage may be from about 1 kV, such as about 5 kV, to about 30 kV. Under the influence of the applied voltage, the substrate and the product from the microreactor move into the open section of the capillary column for separation by electrophoresis. It will be understood that any other suitable means of moving the solution through the column, such as pressure, may be used. Any remaining substrate or product is detected by ultraviolet radiation absorption detection (UV absorption) when it passes through a detection window.

According to yet another aspect of the invention, a device comprising an immobilized-enzyme microreactor in a micropipet tip is provided. This microreactor is prepared by introducing a volume of a sol-gel reaction solution, as further described herein, into a micropipet tip prior to a photopolymerization reaction. The length of the microreactor is controlled by this volume of solution. The solution is then photopoly-merized to form a PSG material and an enzyme is bound to a surface of the PSG material using a procedure similar to that described above.

In use, a solution comprising a peptide substrate or a protein substrate is provided and allowed to flow through the microreactor. The solution may be an aqueous solution comprising the substrate in a buffer. The substrate may be provided in a nanomolar, or sub-micromolar to micromolar concentration range, the selection of which may depend on the tolerances of the detection instrumentation employed. Merely by way of example, sub-micromolar to micromolar substrate concentrations may be suitable when UV absorption is used. The eluate from the microreactor, which comprises the digestion product(s) and any remaining substrate, is collected, whereupon it is separated and analyzed, typically using capillary electrophoresis. Any remaining substrate or product is detected, such as by UV absorption, when it passes through a detection window.

The present invention provides a method for preparing an immobilized-enzyme microreactor. In this method, a PSG material is prepared from a metalorganic monomer and other agents or reagents, such as an organic monomer, a catalyst, a pore template or porogen, and/or a photoinitiator, as further described herein. Various physical properties of the resulting PSG material, or monolith, such as its porosity, pore size, and/or hydrophobicity, can be fine-tuned by adjusting various reaction parameters, such as a molar ratio of the reagents.

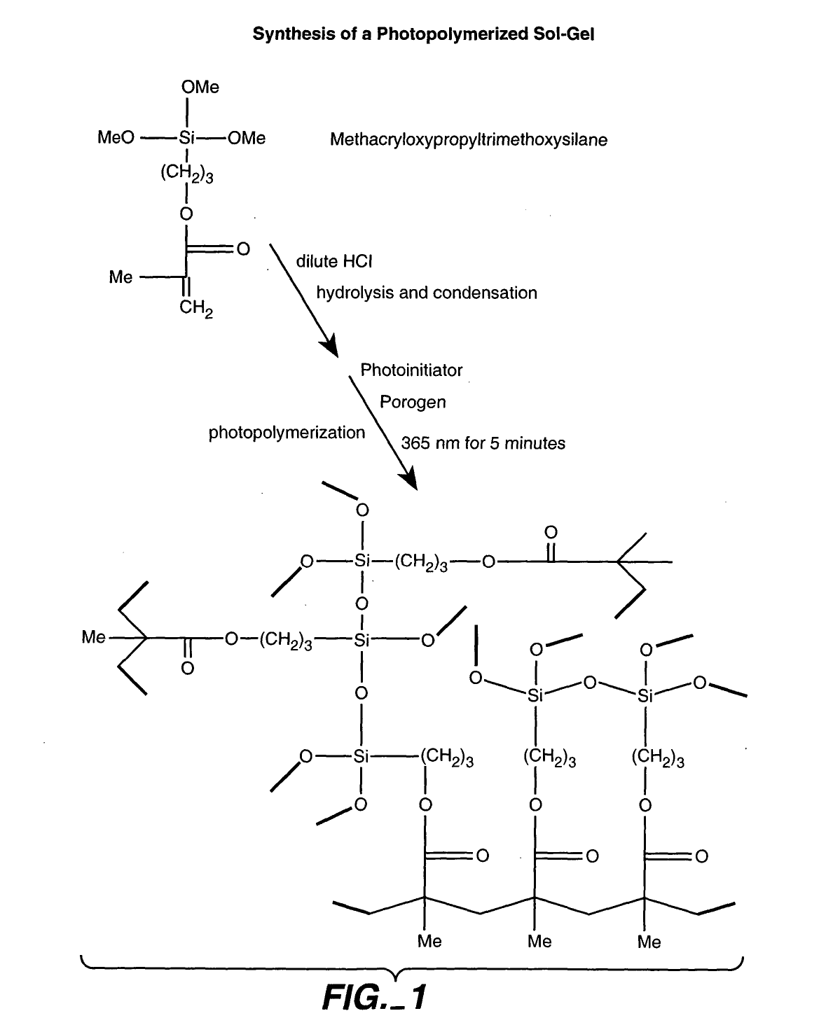

A schematic representation of a synthesis of a PSG material or monolith is shown in FIG. 1. The metalorganic monomer may comprise a metal alkoxide, such as a silane, or a mixture of metal alkoxides. Merely by way of example, the metalorganic monomer shown in FIG. 1 is methacryloxypropyltrimethoxysilane. The metal of the metalorganic monomer may comprise any of, or any combination of, the following metals or metalloids: aluminum, barium, antimony, calcium, chromium, copper, erbium, germanium, iron, lead, lithium, phosphoros, potassium, silicon, tantalum, tin, titanium, vanadium, zinc, and zirconium. The foregoing list of possible metals or metalloids is merely illustrative, not limiting.

A suitable metalorganic monomer may also comprise a photoactive group or a non-hydrolyzable photoactive group, such as a methacrylate group. An example of such a metalorganic monomer is trimethoxysilypropyl methacrylate, also known as methacryl-oxypropyltrimethoxysilane, as shown in FIG. 1. Further, such a metalorganic monomer may be combined with another metalorganic monomer, as exemplified by a combination of methacryloxypropyltrimethoxysilane and bis(triethoxysilyl)ethane or bis(triethoxysilyl)octane, for example.

A metalorganic monomer, such as a metalorganic monomer that either does or does not comprise a photoactive group, may be combined with an organic monomer having such a photoactive group or a hydrophilic organic monomer having such a photoactive group, such as polyethylene glycol dimethyacrylate. Further, a metalorganic monomer may be combined with a photoinitiator, as shown in FIG. 1 by way of example, such as Irgacure 1800, a commercial photoinitiator available from Ciba, Inc. of Tarrytown, N.Y. The photoactive group and/or the photoinitiator facilitates the photopolymerization further described herein.

The metalorganic monomer just described, or the combination of the metalorganic monomer and the organic monomer just described, or the combination of the metalorganic monomer and the photoinitiator just described, is combined with an acid catalyst, such as hydrochloric acid, as shown in FIG. 1 merely by way of example, to partially or completely hydrolyze the metalorganic monomer. This combining, which may and preferably does involve mixing, may take on the order of seconds, such as a few seconds, minutes, such as thirty minutes, or hours, such as twenty-four hours, merely by way of example.

A porogen or a combination of porogens may be combined with the metalorganic monomer, any possible organic monomer, and the catalyst during the above-described hydrolysis process, or may be combined with the above-described combination following the hydrolysis process, to partially condense the metalorganic monomer, as shown in FIG. 1. This combining, which may and preferably does involve mixing, may take on the order of seconds, such as a few seconds, minutes, such as thirty minutes, or hours, such as twenty-four hours, merely by way of example. During this condensation, the metalorganic monomer tends to form dimers, trimers, and/or oligomers of higher orders than trimers. Generally, larger oligomers may be formed at higher reaction temperatures.

The porogen may comprise a solvent or a combination of solvents, such as toluene or a 1:1 mixture of hexane and toluene, for example, a polymer or a combination of solvents, such as poly(methyl methacrylate) or polystyrene, for example, an inorganic salt, such as sodium chloride, which may be in the form of a powder, or sodium sulfate, for example, or any suitable combination thereof. Other suitable porogens include, but are not limited to, benzene, acetonitrile, isooctane, hexane(s), alcohol(s), tetrahydrofuran, acetone, and any suitable combination of any of the above-mentioned possible porogens.

The porogen provides a molecular template for the formation of pores within the porous matrix of the ultimate monolith. The porosity, pore size, and/or pore shape of the porous matrix of the ultimate monolith can be controlled by the type of porogen used and its concentration or volume in the above-mentioned combination. Merely by way of example, a molar or volume ratio of monomer to porogen may be selected and/or adjusted to form desirable pores in the porous matrix of the ultimate monolith, such that the monolith has desirable physical properties, such as porosity, pore size, and/or pore shape.

After the above-described combination has been partially condensed, it is polymerized to form a porous matrix. Preferably, the combination is photopolymerized, as shown in FIG. 1 by way of example. In a photopolymerization process, the combination is irradiated with suitable radiation such that the photoactive group and/or the photoinitiator absorbs the radiation from the radiation source. The photopoly-merization process may take on the order of 5 minutes, as shown in FIG. 1, merely by way of example. The radiation may be visible or ultraviolet light. The wavelength of the radiation is selected in accordance with the type of photoactive group and/or photoinitiator that is used. By way of example, a methacrylate photoactive group may be photopolymerized at a wavelength of about 300 nm to about 365 nm, as reported in C. Yu et al., Electrophoresis 2000, 21, (1) 120-127 and H. G. Woo et al., Bull. Korean Chem. Soc 1995, 16, 1056-1059. Further by way of example, the Irgacure 1800 photoinitiator may be photopolymerized at a wavelength of about 365 nm. Absorption of the radiation starts a photochemical reaction that catalyzes the polymerization of the metalorganic monomer, and/or associated dimers, trimers, and/or oligomers. The photopolymerization results in the formation of a relatively homogeneous porous matrix. An example of a product of a photopolymerization process is shown in FIG. 1.

PSG monoliths useful in the present invention are generally easy to produce, as demonstrated above, have high porosity, and can be chemically modified. A suitable PSG monolith may have a pore size in the range of 0.5 μm to about 20 μm in diameter, on average, by way of example. Advantageously, the porous characteristics of such PSG monoliths, such as the size, shape, and number of pores in the polymer matrix of the monolith, may be controlled or adjusted by a number of variables, such as the amount of cross-linking monomer employed, the quality of the porogenic solvent employed, and/or the molar ratio of the monomer and the porogenic solvent employed.

According to one example, a metalorganic monomer and a hydrophilic organic monomer, such as polyethylene glycol dimethacrylate, are mixed for 30 minutes with an acid catalyst, such as hydrochloric acid, to promote hydrolysis and condensation of the metalorganic monomer. A photoinitiator, such as Irgacuare 1800, is added to the resulting solution, and the combination is stirred for 30 minutes at room temperature, or about 20° C. to about 25° C. To the resulting combination, an appropriate volume (such as 70% to 90% of the total solution volume) of a porogen template is added. The resulting sol-gel solution is now ready for photopolymerization, such as that described previously.

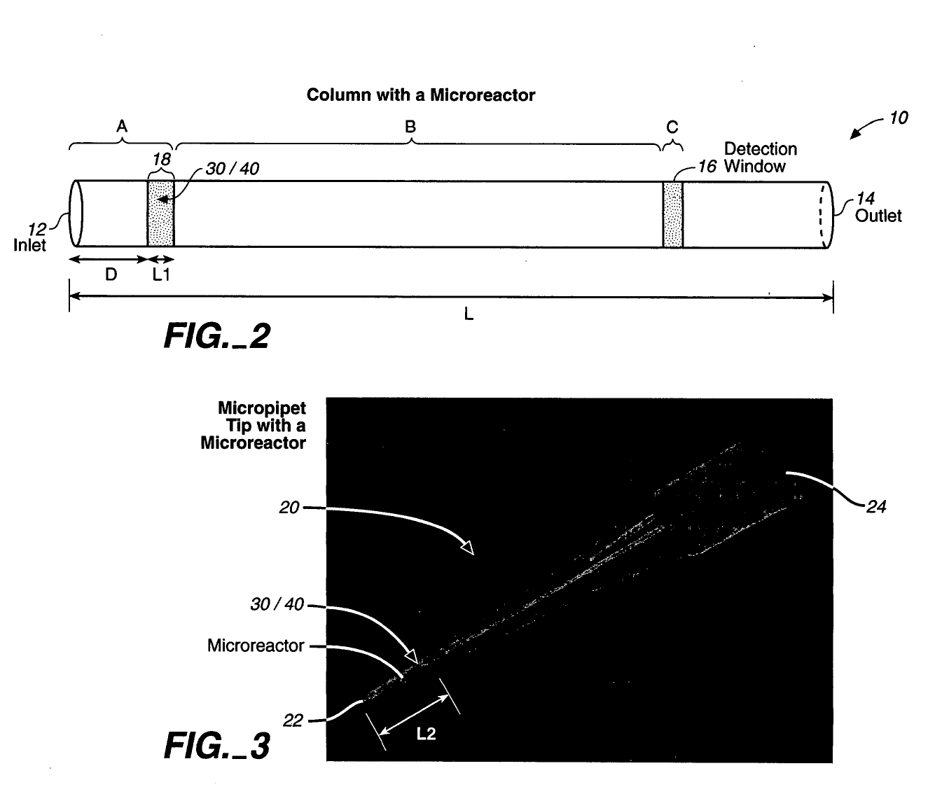

According to one application, the PSG material or monolith may be prepared in a capillary column 10, as shown in FIG. 2 by way of example. In such an application, a suitable capillary column 10 is provided, such as a capillary column 10 having an inner diameter of about 75 μm, an inlet end 12 and an outlet end 14, and an overall length L therebetween of about 20 to about 30 cm, merely by way of example. The capillary column 10 typically has a sample introduction and reaction area A near the inlet 12, an intermediate separation area B for separation of one or more analyte(s) in a sample that is introduced to the column, and an ultimate detection area C for detecting any such analyte near the column outlet 14. Merely by way of example, a distance between the inlet 12 and the beginning of the detection area C may be on the order of about 15 cm to about 20 cm, and a distance between the beginning of the detection area C and the outlet 14 may be on the order of about 5 cm to about 10 cm. The detection area C may comprise a detection window 16, as shown in FIG. 2, through which the sample analyte(s) can be detected.

A sol-gel solution 30, such as that described in the example above, is introduced into a suitable capillary column 10 and the column is sealed at its inlet end 12 and its outlet end 14. Any suitable sealing means or method (not shown) may be used, such as the use of removable seals, merely by way of example. The sol-gel solution 30 is typically introduced via the inlet 12 of the capillary 10 at some distance D away from the inlet, as shown in FIG. 2. This distance D is typically selected simply as a matter of design convenience, and may be on the order of millimeters (about out 3 mm to about 5 mm, for example) or a few centimeters (about 1 cm to about 3 cm, for example), merely by way of example.

The capillary column 10 may have an outside radiation-blocking coating (not shown), such as a polyimide coating. In such a case, a section of the coating is removed to create a radiation-transparent window 18 through which radiation may be passed to photopolymerize the sol-gel solution. Where the coating remains, radiation is blocked and the sol-gel solution does not photopolymerize. Alternatively, the capillary column may have radiation-transparent walls. In such a case, the column may be covered with a radiation-blocking material to block radiation exposure where desired, while leaving a radiation transparent window 18. In either case, the length of the radiation-transparent window 18 of the capillary controls the length L1 of the PSG material in the column. The length L1 may be on the order of from about 1 cm to about 10 cm, merely by way of example, and may be selected as appropriate relative to the dimensions of the column 10 and/or relative to the particular application.

After the sol-gel solution 30 is introduced in the capillary 10 and the radiation transparent window 18 is appropriately provided, the capillary 10 is exposed to suitable radiation for a time sufficient for formation of the monolith. Merely by way of example, the capillary may be exposed to ultraviolet radiation, at a wavelength of about 360 nm to about 365 nm, or about 365 nm, for about 5 minutes, as described above in relation to FIG. 1. The capillary is then unsealed and rinsed with an organic solvent, such as ethanol or acetonitrile, to remove any unreacted sol-gel solution from the capillary column. The capillary is now ready for further processing, as further described herein.

According to another application, the PSG material or monolith may be prepared in a micropipet tip 20, as shown in FIG. 3 by way of example. In such an application, a certain volume of a sol-gel solution 30, such as that described in the example above, is taken up into the micropipet tip 20, whereupon the tip is sealed at its proximal end 22 and its distal end 24. Any suitable sealing means or method (not shown) may be used, such as the use of seals merely by way of example. For a given micropipet tip 20, this volume of solution 30 defines the length L2 of the PSG material in the tip. The micropipet tip 20 may be of any suitable volume, such as from about 0.5 microliters to about 200 microliters, merely by way of example. In a 200-microliter micropipet tip 20, the PSG length L2 may range from about 3 mm to about 8 mm, further by way of example.

The filled micropipet tip 20 is then exposed to suitable radiation for a time sufficient for formation of the monolith. Merely by way of example, the micropipet tip 20 may be exposed to UV radiation, at a wavelength of about 360 mm to about 365 nm, or about 365 nm, for about 5 to about 10 minutes, as described above in relation to FIG. 1. The tip 20 is then unsealed and the monolith is rinsed with an organic solvent, such as ethanol or acetonitrile, to remove any unreacted sol-gel solution. The micropipet tip is now ready for further processing, as further described herein.

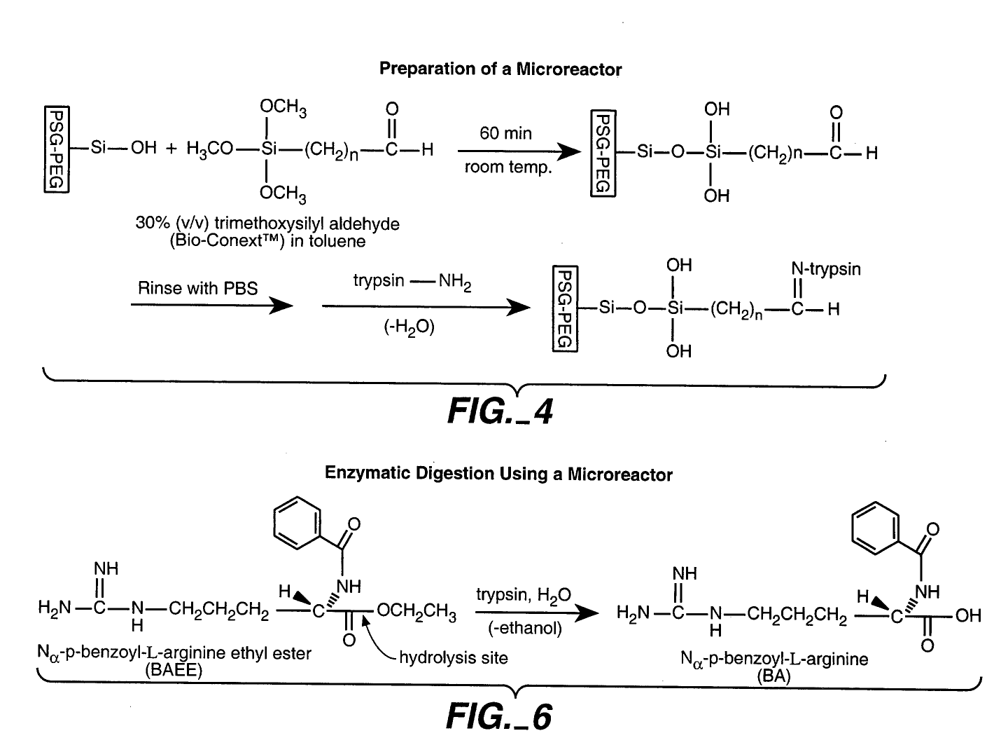

According to the present invention, a surface of a PSG material or monolith, such as any of those described above, is derivatized with a linker, or a reagent, comprising a functional group that facilitates the binding of an enzyme to the surface. An example of such a derivatization is shown in FIG. 4 by way of example, wherein PSG-PEG refers to a PSG material that results from a combination of a metalorganic monomer with a hydrophilic organic monomer, such as polyethylene glycol (PEG) dimethacrylate, as described above. Any suitable linker having a suitable functional group may be used, and any suitable enzyme may be attached to the PSG monolith. By way of example, the reagent may be a silicate reagent comprising an aldehyde functional group that facilitates the covalent bonding of an enzyme having an active amine functional group, such as trypsin, for example, to the surface of a PSG material, as shown in FIG. 4. Further by way of example, the silicate reagent may comprise a trialkoxy silane comprising a non-hydrolyzable aldehyde group, such as a trimethoxysilyl aldehye, as shown in FIG. 4.

In the derivatization process, a PSG material is rinsed free of water with a rinsing agent, such as an organic solvent, for example. By way of example, suitable solvents include acetonitrile and toluene, the latter being preferred. A solution comprising a derivatizing reagent, such as the above-described silicate reagent, at an appropriate concentration, such as about 30 volume percent relative to the solution, is prepared. This solution is applied to the PSG material and is allowed to flow through the PSG material under conditions sufficient for suitable derivatization, such as at a temperature of about 4° C. to about 23° C., or room temperature (from about 20° C. to about 25° C.), for a period of about 30 to 60 minutes, merely by way of example, as shown in FIG. 4. This flow may be accomplished via normal flow of the solution relative to the PSG material, via pumping of the solution relative to the PSG material, via placing the PSG material in the solution, which may encompass incubation, merely by way of example. At the end of the period, the PSG material is rinsed with a rinsing agent, such as toluene, for example, to stop the reaction and to remove any unreacted reagent and any alcohol by-product.

In the enzyme-bonding process, the PSG material is conditioned with a conditioning agent, such as a buffer solution that will not interfere with the bioactivity of the enzyme. An example of a suitable buffer solution is a phosphate buffered saline (PBS) solution (about 50 mM, for example), as shown in FIG. 4 by way of example, that contains a high concentration of calcium chloride (about 200 mM, for example) to prevent self-destruction of the enzyme. A dilute enzyme-buffer solution (about 10 mM to about 100 mM, for example) is applied to the PSG material and allowed to flow through the PSG material. An example employing a trypsin enzyme, and resulting in an immobilized-enzyme PSG material, is shown in FIG. 4 by way of example.

A derivatized PSG material 40 may be incorporated into a microreactor device, such as the integrated microreactor-column 10 (FIG. 2) or the integrated microreactor-micropipet 20 (FIG. 3) described above. When a derivatized PSG material 40 is used in a microreactor device, the ends of the device are sealed, for example, using sealing devices, and the reaction between the amine group of the enzyme and the aldehyde group at the surface of the PSG material is allowed to proceed at a low temperature, such as from about 4° C. to about 40° C. for several hours, such as from 30 minutes to about 24 hours, such as from about 15 hours to about 24 hours. Higher temperatures may be used to shorten the reaction period, although care should be taken to avoid temperatures that result in the autolysing, self-destruction, or degradation of the enzyme. Appropriate temperatures generally depend on the nature of the enzyme. The microreactor device is then unsealed, for example, by removing any sealing devices, and any unbound trypsin is removed from the device by flowing copious amounts of phosphate buffered saline through the PSG material.

It will be understood that any enzyme suitable for immobilization on a PSG material and for use in a separation process are contemplated herein. As mentioned above, trypsin, pepsin, and chymotrypsin are merely examples. Further examples include, but are not limited to, malate dehydrogenase, citrate lyase, isocitrate dehydrogenase, and lactate dehydrogenase, which can be used in various assays, such as assays of organic acids. By way of example, malate dehydrogenase, citrate lyase, isocitrate dehydrogenase, and lactate dehydrogenase can be used for the enzymatic analysis of oxaloacetate, citrate, D-isocitrate or succinate, and lactate or pyruvate, respectively. It will be appreciated that there are numerous enzymes that could be immobilized relative to a PSG material as contemplated herein, and numerous resulting immobilized-enzyme PSG materials that could be employed in separation processes, such as biomolecule separation processes involving the separation of one or more proteins, oligonucleotides, peptides, steroids, organic acids, or any combination thereof, by any suitable separation means.

Additionally, it will be understood that any means of immobilizing the enzyme relative to the PSG material, and any combination of such means, are contemplated herein. As mentioned above, a flow-through technique, wherein an enzyme solution is flows through a PSG material and the immobilization is allowed to proceed at a low temperature for a number of hours, is one example. Merely by way of example, a further example is a pump-through technique, wherein an enzyme solution is pumped through a PSG material and the immobilization is allowed to proceed at a low temperature for a suitable time, such as a number of hours. Yet another example is incubation, such as incubation of a PSG material in an enzyme solution at an appropriate temperature for an appropriate time, such as about 4° C. to about 40° C. for about 30 minutes to about 24 hours, such as about 15 hours to about 24 hours.

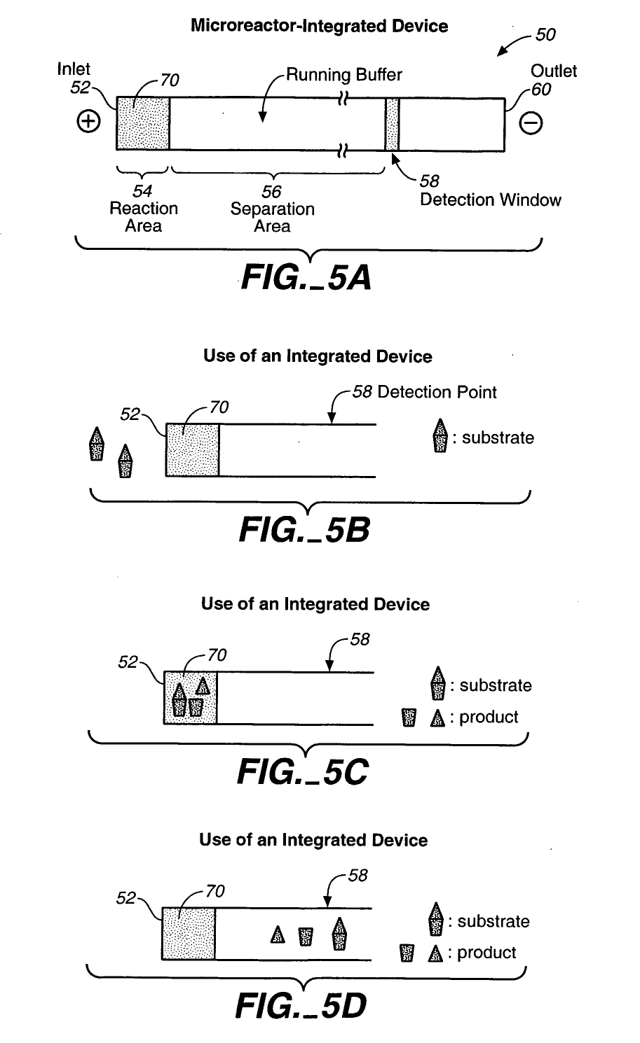

As described above, an immobilized-enzyme microreactor may be incorporated into a device, such as a capillary column or a micropipet tip, for example. A microreactor-integrated CE column 50 is schematically shown in whole or in part in FIG. 5. Near the inlet 52 of the column 50 is a reaction area 54, which comprises a microreactor 70 according to the present invention, as shown in FIG. 5A. Downstream relative to the microreactor 70 is a separation area 56, and downstream relative to the separation area 56 is a detection area or window 58, which is upstream of the column outlet 60, as shown.

As mentioned in connection with the integrated capillary column of FIG. 2, there may be an open section, such as a short open section on the order of a few millimeters long, located between the inlet 52 and the reaction area 54, or microreactor 70, although ideally, no such open section is employed. If an open section is employed, as it may be for practical reasons such as to facilitate the handling of the column 50 and/or to avoid damage to the reaction area 54 or microreactor 70 when the column is cut, as it often is when used in connection with a capillary cartridge of an analytical instrument, such as a Beckman P/ACE cartridge (Beckman Coulter, Inc., of Fullerton, Calif.), its length is preferably minimized.

In use, a voltage is applied to the column, as represented by a circled positive (+) sign at the column inlet 52 and a circled negative (−) sign at the column outlet 60, merely by way of illustration, to facilitate electroosmotic flow from the inlet to the outlet. As shown in FIG. 5B and FIG. 5C, a sample comprising one or more analyte(s) or substrate(s) is applied to the column 50 via the inlet 52, typically using an introduction voltage, such as a voltage on the order of a few kV, and allowed to flow to the microreactor under the influence of the applied voltage, which may be on the order of about 10 kV, for example, whereupon an enzymatic digestion reaction occurs by way of the microreactor's immobilized-enzyme PSG monolith, as described herein. As shown in FIG. 5C, as this digestion reaction proceeds, both the substrate and one or more product(s) of the digestion reaction are present in the reaction area A, or the microreactor 70.

It is possible to determine the distance that the substrate bands needs to travel relative to an integrated device, such as the column 50, in order to overlap or be in the immobilized-enzyme microreactor section of the device, based on electroosmotic flow, for example. When the substrate band has traveled the required distance or is otherwise in the correct position relative to the microreactor, the enzymatic reaction is allowed to proceed for a certain amount of time, such as from about 5 seconds to about 60 minutes, such as from about 5 seconds to about 15 minutes, merely by way of example. The amount of time may vary according to the reaction conditions, such as temperature.

As a running buffer solution is applied to the column, the substrate and the reaction product(s) move downstream into the separation area 56 and on to the detection area 58, as shown in FIG. 5D, whereupon they are detected via detection window 58 by any suitable detection means. Merely by way of example, the detection area 58 may be exposed to radiation, such as ultraviolet radiation, for example, via the detection window 58, and a signal relating to radiation absorption can be detected. The detected signal may then be read or manipulated to obtain a useful qualitative or quantitative result that relates to the amount of any substrate or any reaction product passing through the column over time.

### EXPERIMENTAL EXAMPLES

Experimental examples pertaining to the present invention are provided below, starting with the following experimental example.

Reagents and Chemicals. All buffers and solutions were prepared using a Milli-Q water purification system from Millipore (Billerica, Mass.) and degassing by sonication for 5 minutes prior to use. Methacryloxypropyltrimethoxysilane (MPTMS), polyethylene glycol di-methacrylate with an average molecular weight of 330 Daltons (PEG-DM), toluene, N_{α}-L-arginine ethyl ester (BAEE), N_{α}-benzoyl-L-arginine (BA), bradykinin, and phosphate buffered saline (PBS) were purchased from Sigma-Aldrich (Milwaukee, Wis.) and used as received. BAEE, BA, and bradykinin were all dissolved in 50 mM TRIS-HCl (pH 7.4 or 7.5) buffer for analysis. Irgacure 1800 was received from Ciba, Inc. (Tarry-town, N.Y., USA).

Typsin-PSG Microreactor Preparation. A 30-cm-long, fused-silica capillary column was obtained from Polymicro Technologies (Phoenix, Ariz.). The capillary column was 75-μm in inner diameter and 365-μm in outer diameter and was coated with polyimide. At a point at approximately 5 mm from the inlet of the capillary, a 1-cm-long stripe of polyimide was removed from the outside of the capillary column using a razor blade to provide an irradiation window.

The parent PSG monolith was prepared using Solution A and Solution B. The PSG reaction solution was prepared with Solution B. Solution A was made by mixing 400 mL of MPTMS, 175 μL of PEG-DM, and 100 μL 0.12 N HCl at room temperature and in the dark for 30 min. Solution B was made by first dissolving 40 mg of Irgacure 1800 in 320 μL of toluene followed by the addition of 80 μL of Solution A, which was stirred at room temperature and in the dark for 5 minutes. The above-mentioned capillary column with the 1-cm irradiation window near its inlet was flushed with approximately 200 μL of Solution B. The ends of the capillary were sealed with parafilm plugs and the sealed capillary was irradiated in a Spectrolinker UV crosslinker oven (Spectronix Corp.) with predominantly 365 nm bulbs for 5 minutes. The capillary was unsealed, and unreacted starting materials were pressure-rinsed from the capillary columns using a syringe filled with ethanol. Any monolith columns that were not used were rinsed and stored in acetonitrile.

The parent monolith was derivatized with triethoxysilyl aldehyde (BioConext from United Chemical Technologies, Inc.) or with triethoxysilyl butraldehyde (Gelest, Inc.). In this process, the parent PSG monolith was rinsed with toluene to remove as much water in the capillary column as possible. After rinsing with toluene, approximately 100 μL of a 30% by volume solution of the alkoxysilane reagent (referred to as the “linker”) in toluene was allowed to flow through the 30-cm capillary column for 30 to 60 minutes at room temperature. The alkoxy groups of the linker reacted with free silanol groups on the PSG monolith. Any unreacted linker was removed from the capillary column by rinsing the column with toluene, followed by acetonitrile, if the derivatized PSG monolith was not further reacted with an enzyme solution.

An aldehyde-derivatized PSG monolith was prepared for enzyme immobilization by first flushing the column with 10 mM phosphate-buffered saline (PBS). A freshly prepared solution of trypsin (10 mg/mL) in PBS was then allowed to flow through the capillary column. Approximately 100 μL of the trypsin solution flowed through the capillary. The ends of the capillary were then capped and the enzyme-filled capillary was allowed to sit overnight in a refrigerator at 4° C. overnight (about 17 hours) after which the capillary was flushed with PBS and either stored at 4° C. or loaded into a Beckman P/ACE column cartridge for immediate use.

Multiple immobilized-enzyme, microreactor capillary columns were prepared as described above. When any such capillary column was not put to use, it was stored at 4° C.

The capillary column was checked to see that no unbound trypsin was left in the column. In this determination, the enzymatic activity of the rinse liquid, or the PBS that ran through the column, was checked for the presence of trypsin using BAEE. BAEE was allowed to react with any trypsin that might be left in the rinse liquid. If trypsin was present in the rinse liquid, it reacted with BAEE to produce the enzymatic product, BA, which was then detected in an analysis of the BAEE-rinse-liquid solution by capillary electrophoresis. If BA was no longer detected by CE, it was determined that all of the unbound trypsin was removed from the capillary column. This determination was performed to ensure that any enzymatic reaction occurring in the column involved immobilized or covalently bound trypsin, rather than free trypsin still present in the column.

Trypsin-PSG Device Operation. A trypsin-PSG capillary was installed in a Beckman P/ACE 2000 capillary cartridge (Beckman Coulter, Inc., of Fullerton, Calif.), whereupon about 4.4 cm of the original 30-cm long column was cut off. The total length of the resulting capillary column was about 25.6 cm, with the length from the detection window to the outlet being about 6.7 cm. The 1-cm enzyme microreactor region was approximately 5 mm from the capillary inlet. Prior to operation, the trypsin-PSG capillary column was conditioned by rinsing it with a separation buffer using a 0.5-mL disposable syringe in a manual vise. The separation buffer was 50 mM TRIS (pH 7.4). Electrokinetic conditioning of the column followed using a Beckman P/ACE 2000 CE instrument (Beckman Coulter, Inc., of Fullerton, Calif.) at 5 kV for 30 min.

Multiple trypsin-PSG capillaries were prepared as described above, although the lengths of the capillaries varied. CE or CEC separation conditions varied depending on the length of the microreactor. Typically, an electrokinetic injection of about 5 kV or less was used to put the sample on the column. Separation voltages of from about 8 kV to about 10 kV were typical. Substrate and product(s) were detected at 214 nm at run temperatures of 20° C.

BAEE at a concentration of 2.5 mM was used as an artificial substrate to demonstrate the activity of the microreactor, as described below. Also, bradykinin at a concentration of 1 mg/mL in water was used to demonstrate the effectiveness of the microreactor in the digestion of a peptide, as further described below.

Determination of bound trypsin. A trypsin-PSG monolith, as described above, may be exposed to a rinse liquid of 100 mM NaOH to degrade the PSG monolith and thus remove it from the column. In such a process, the trypsin is generally not degraded, although its three-dimensional structure may no longer be intact. The amount of trypsin that was once bound to the PSG surface may then be determined using a Bradford Assay, which utilizes Coumassie Blue dye. This determination is not affected by whether the three-dimensional structure of the trypsin is intact or not. The resulting trypsin-dye complex may be detected and quantitatively measured by absorption spectroscopy. Such determinations may be used to determine the amount of an enzyme, such as trypsin, that is immobilized or covalently bound to the surface of an immobilized-enzyme PSG monolith.

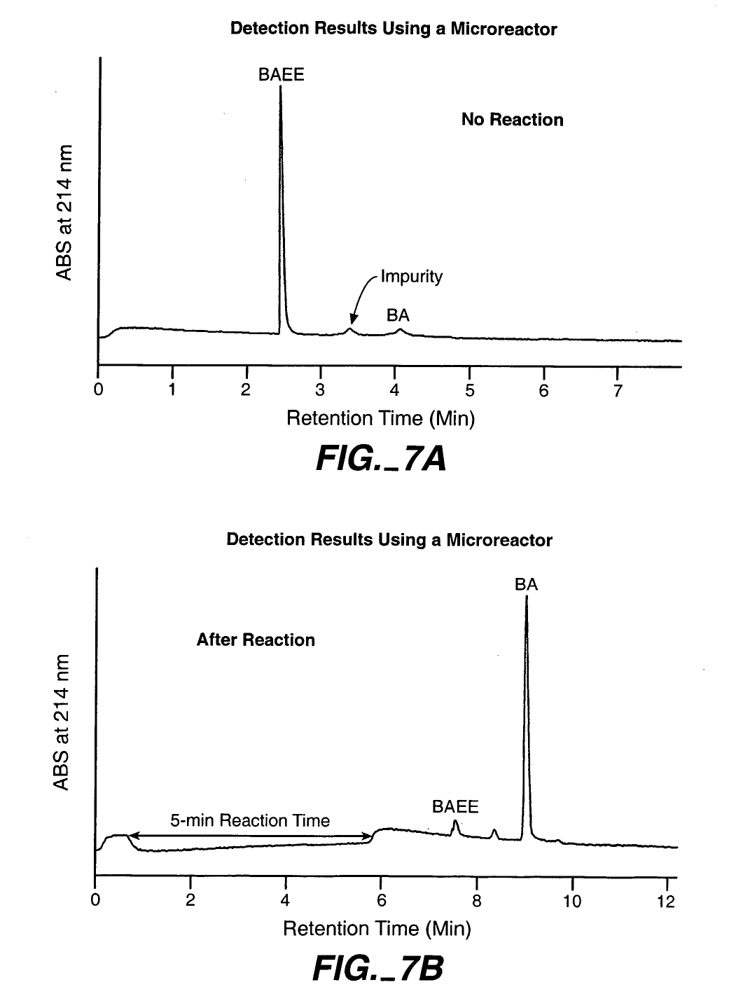

Results. A column incorporating a 1-cm immobilized-trypsin PSG, such as that described above in relation to FIG. 2, was used to cleave across the ester bond of BAEE. This cleavage resulted in the formation of ethanol and BA, as schematically shown in FIG. 6. The 20 mM BAEE (BAEE dissolved in 50 mM TRIS-HCl buffer (pH 7.5)) was injected into the column over 2 seconds at 2 kV, a voltage of 10 kV was applied to the column, and the BAEE was digested at a temperature of 20° C. over various periods. The absorption data collected over time (ABS at 214 nm versus Retention Time in minutes) in this experiment is graphically represented in FIG. 7A and FIG. 7B, where the data of FIG. 7A relates to the BAEE sample prior to digestion, and the data of FIG. 7B relates to the sample after digestion on the column. From this data, it was determined that the reaction at 20° C. for about 5 minutes was sufficient for the total, or near total, digestion of BAEE.

It was further determined that there was an approximately linear relationship (y=0.0004+0.0003; R²=0.9903) between the amount of time the BAEE spent on the column and the amount of product formed, as reflected by absorption at 214 nm. That is, at least the initial rate of the reaction up to about 5 or so minutes is approximately linear.

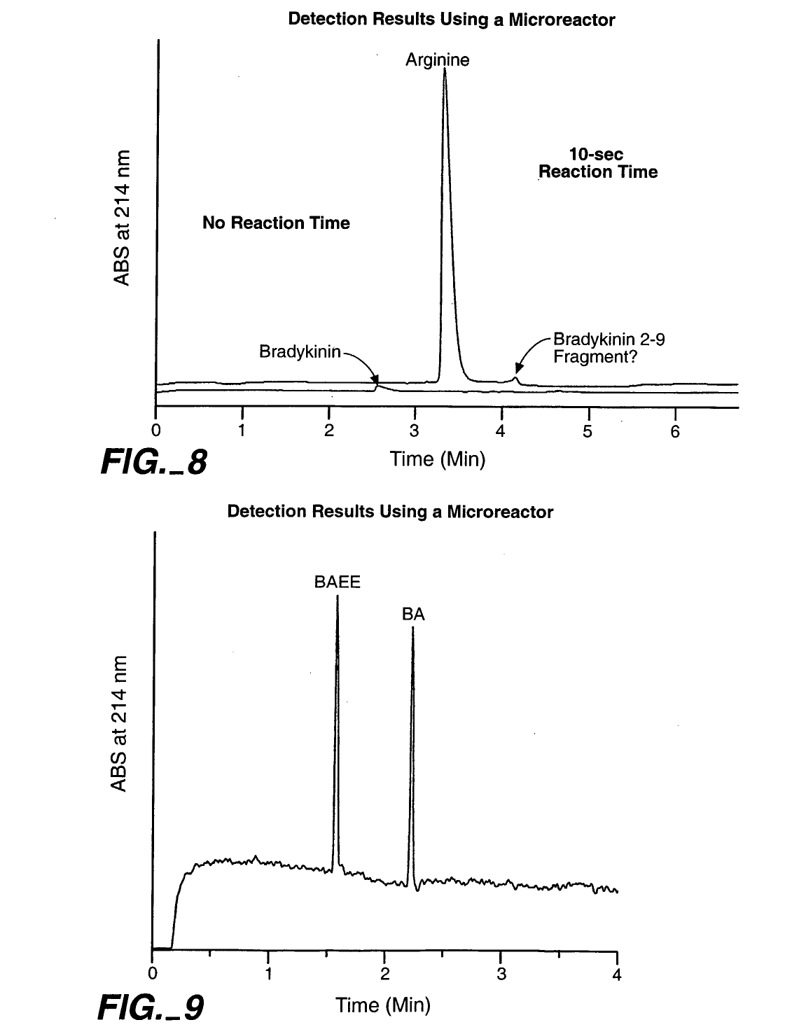

The activity of trypsin was also demonstrated by cleaving the peptide bradykinin. The bradykinin (1 mg/mL) was introduced into a column incorporating a 1-cm immobilized-trypsin PSG, in the manner described above. The bradykinin was digested, resulting in the digestion reaction product, arginine. The bradykinin and the arginine were separated, as demonstrated in the graphical representation shown in FIG. 8, where the lower “curve” of the figure relates to the bradykinin sample prior to digestion, and the upper “curve” relates to the sample after digestion on the column. From this data, it was determined that the reaction at 20° C. for within 20 seconds, such as about 10 seconds, was sufficient for the total, or near total, digestion of the bradykinin. This is a very short digestion time.

As shown in FIG. 8, little of the bradykinin and little of its fragment were detected. One possible explanation for this result is that both bradykinin and arginine bind electrostatically to the negatively charged PSG monolith, with arginine being attracted to the PSG surface much less strongly than bradykinin. If a different buffer is used, such as a buffer of higher concentration than the one used in this example, or a buffer with a micromolar concentration of NaCl, by way of example, it may be possible to avoid the electrostatic interaction between the negatively charged PSG surface and the bradykinin and the arginine. However, care should be taken to ensure that any such buffer does not interfere with the enzymatic reaction. Other means of modifying the parent PSG monolith such that its surface is positively rather than negatively charged, may be employed, provided the sources of the negative charges, namely, the free silanol groups of the monolith, are sufficient to serve this purpose as well as the purpose of interacting or covalently bonding with the linker.

The experimental example just described is notable for demonstrating that the enzyme, trypsin, is able to cleave protein fragments, and is able to do so quite efficiently. The example is more particularly notable for demonstrating that such an enzyme, as used in connection with a PSG monolith according to the present invention, effectively cleaves protein fragments. In assessing the results, it should be noted that all of the digestion reactions just described were performed at 20° C., which is far from the optimal temperature of 37° C. for activity of the trypsin enzyme. It is contemplated that running the digestion reactions at a higher temperature, such as a temperature approaching or reaching 37° C., would further demonstrate that an enzyme-PSG monolith microreactor is very effective for the digestion of biological samples and their components.

In another experimental example, a first test was performed in which BAEE was enzymatically digested at about 20° C. using trypsin in bulk solution in a CE column. Any remaining BAEE and any product(s) of the digestion reaction were detected via UV absorption. Absorption data was collected over time (ABS at 214 nm versus Retention Time in minutes), and the peak height for BA was used to determine the amount of BA digestion product produced. In a separate test, BAEE was enzymatically digested at about 20° C. using an immobilized-trypsin microreactor that was incorporated into a CE column, according to the present invention. As in the first run, absorption data was collected over time and the height of the absorption peak associated with the BA was used to determine the amount of BA produced. When the results of the two tests were compared, it was determined that there was about a 200-fold increase in the peak height associated with the BA product when the trypsin-immobilized microreactor was employed. This result demonstrates that the bioactivity of the enzyme, trypsin, may be enhanced up to 200-fold by being immobilized onto a PSG material according to the present invention. It is contemplated that running the digestion reactions at a higher temperature, such as a temperature approaching or reaching 37° C., or the optimum temperature for the activity of the trypsin enzyme, would further demonstrate that an enzyme-PSG monolith microreactor is very effective for the digestion of biological samples and their components. Further, it is contemplated that even greater enhancements are possible when using this and other enzymes according to the present invention.

In yet another experimental example, an immobilized-trypsin microreactor was prepared in a micropipet tip, more or less as described herein in relation to FIG. 3. The micropipet tip was made of plastic and had a volume capacity of about 200 μL. Approximately 100 μL of 20 mM BAEE (BAEE dissolved in 50 mM TRIS-HCI buffer (pH 7.5), for example) was loaded onto the top of the microreactor. Pressure was applied to the micropipet tip using a 200-μL automatic pipet to allow the BAEE solution to flow through the micropipet tip within a period of about 10 minutes. The applied pressure was relatively low and the flow rate was crudely estimated (based on the known volume of BAEE solution applied and the overall period of flow) at about 10 μL per minute. More accurate determinations of flow rate within the microreactor are possible and contemplated. After the 10-minute period of flow, at a temperature of about 22° C., the eluate was evaluated for digestion product by injecting the eluate into an empty 25.6-cm-long CE capillary that had in inner diameter of 75 μm, and running a separation buffer of 50 mM TRIS-HCL (pH 7.5) through the column at a temperature of about 20° and an applied voltage of about 10 kV. Any remaining BAEE and any product(s) of the digestion reaction were detected via UV absorption. Absorption data was collected over time (ABS at 214 nm versus Retention Time in minutes), as shown in FIG. 9, and the peak height for BA was used to determine the amount of BA digestion product produced.

The results of this experimental example, as well as other results described herein, demonstrate that when a microreactor is used in connection with a micropipet tip, or with a column, according to the present invention, the immobilized enzyme, trypsin, is able to cleave protein fragments. These are promising results, in view of the fact that, as demonstrated above, the bioactivity of the enzyme, trypsin, may be enhanced up to 200-fold by being immobilized onto a PSG material according to the present invention, even when employed at a temperature that is less than optimum for the activity of the enzyme. It is contemplated that enhancements beyond any associated with the Experimental Examples provided herein may be achieved according to the present invention. Further, it is believed that results, such as those associated with these Experimental Examples, would flow from the use of other enzymes, such as any described or contemplated herein, in connection with a microreactor and devices incorporating same, according to the present invention.

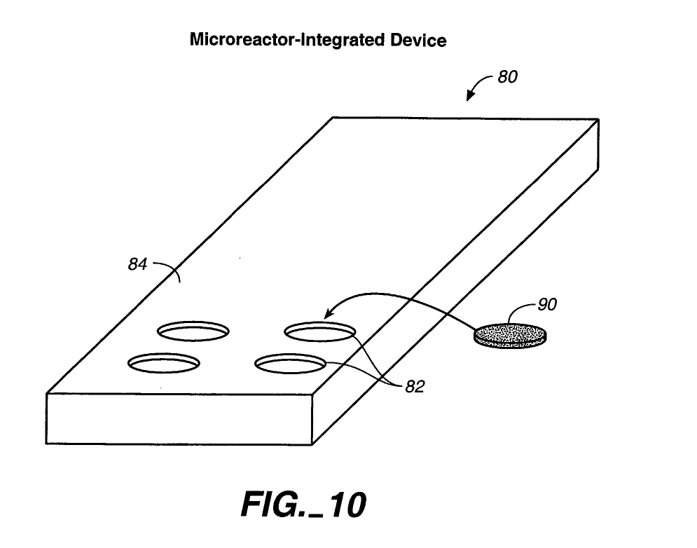

Accordingly, the present invention encompasses an immobilized-enzyme microreactor and any such microreactor that may be incorporated into any number of appropriate devices, such as a capillary column or a micropipet tip, as described above, in addition to other devices. Merely by way of example, an integrated microreactor device 80 that comprises a microreactor 82 incorporated into a well-plate device is contemplated herein, as partially shown in FIG. 10. In such an integrated microreactor device 80, a microreactor 90 according to the present invention is introduced or inserted in one or more well(s) 82 of a well plate 84, as partially shown in FIG. 10. Often there are 96 wells 82 in a typical well plate 84, typically arranged in one or more row(s) and column(s), although a plate with one well to any number wells and any suitable arrangement is contemplated herein. The wells 82 comprise indentations, which are typically circular in shape, in the well plate 84, which is typically rectangular in shape and composed of plastic. Any other suitable shape or composition associated with the well(s) 82 or the well plate 84 is contemplated. The microreactor 90 may be formed in any particular shape or dimension to accommodate or to correspond with the shape or dimension of the well 82, as desired or necessary, such as in the shape of an appropriately dimensioned circular plug, as shown by way of example in FIG. 10, such that it can be easily introduced and/or fit, such as close-fit, for example, into the well 82. Once a microreactor 90 is introduced into a well 82, as schematically represented by an arrow in FIG. 10, the integrated microreactor device 80 can be used for enzymatic digestion of samples applied thereto, in a manner such as that previously described.

The invention encompasses a great variety of variations and modifications. For example, the device of the invention, or an integrated device of the invention, may contain one or more enzyme(s) immobilized on one or more PSG material(s), and/or multiple enzymes co-immobilized on one or more PSG material(s), such as in series, for example, for the digestion of a solution containing more than one peptide and/or protein substrate. Immobilization of an enzyme according to the present invention reduces or substantially prevents self-destruction of the enzyme, such that the enzyme is effective in the microreactor device.

Further by way of example, the surface area and pore volumes of the PSG material can be modified to accommodate proteins that are large in size, such as proteins having molecular weights of about 200 Daltons or more, such as 200 kDaltons or more, and/or being on the order of more than nanometer size. The PSG material can also be used in connection with a substrate that is much smaller in molecular weight and/or size. As yet further examples, the immobilized-enzyme microreactor and its preparation can be adapted to devices that comprise well-plates and/or that have shapes other than the shape of a capillary or a micropipet tip.

The present invention provides an enzyme that is immobilized by covalent bonding to the surface of a porous PSG material that is versatile in application. For example, the immobilized-enzyme, porous PSG material, or monolith, can be used in a variety of formats, such as a microreactor, a capillary or a micropipet format, as well as other formats. The immobilized-enzyme, porous PSG material provides a suitable, if not ideal, interface for downstream analysis applications and instrumentation, such as a variety of separation applications and tools including HPLC, CEC, and CE, for example. In another example, the immobilized-enzyme, porous PSG material provides an interface for separation by mass spectrometry, wherein the reaction products of the enzymatic digestion process are fed into mass spectrometer using an electrospray ionization interface, for example.

The immobilized-enzyme, porous PSG material is particularly useful in the separation of proteinaceous and/or peptidic analytes from sample, and may be designed to accommodate such analytes that are relatively large. In a particular application, the bioactivity of the enzyme, trypsin, was enhanced up to 200-fold by being immobilized onto a PSG material, even when the temperature employed was less than the optimal temperature of the activity of that enzyme. Such an immobilize-enzyme, PSG material is thus particularly useful in biomolecule separations, such as separations of one or more proteins, oligonucleotides, peptides, steroids, organic acids, or any combination thereof.

Immobilization of an enzyme with respect to a PSG material, as described herein, can result in an increase in the activity of the enzyme by a several or many orders of magnitude. It is believed, without being so bound, that in the immobilization process, the enzyme becomes covalently bound to the surfaces in the pores of the PSG material. When the analyte, or substrate, enters a pore, its concentration in the pore relative to its concentration in the bulk of the material, is greatly increased. As the activity of the enzyme depends on the substrate concentration, typically in direct proportion, it too is greatly increased. As such, the immobilize-enzyme, PSG material of the present invention provides a highly active and effective tool for the breakdown, separation, and analysis of biomolecules and their components.

Various references, publications, provisional and/or non-provisional United States patent applications, and/or United States patents, have been identified herein, each of which is incorporated herein in its entirety by this reference. Various aspects and features of the present invention have been explained or described in relation to understandings, beliefs, theories, underlying assumptions, and/or working or prophetic examples, although it will be understood that the invention is not bound to any particular understanding, belief, theory, underlying assumption, and/or working or prophetic example. Various modifications, processes, as well as numerous structures to which the present invention may be applicable will be readily apparent to those of skill in the art to which the present invention is directed, upon review of the specification. Although the various aspects and features of the present invention have been described with respect to various embodiments and specific examples herein, it will be understood that the invention is entitled to protection within the full scope of the appended claims.

1-84. (canceled)

85. A method comprising:
providing a polymerized sol-gel material (PSG); and
linking an enzyme to a surface of the PSG via covalent linkage.

86. The method of claim 85, wherein the providing comprises providing the PSG in a device selected from a microreactor, a column, a pipet, a well, and a well plate.

87. The method of claim 86, wherein the device is sufficient to separate an analyte from a sample.

88. The method of claim 85, wherein the providing comprises providing a PSG that comprises a photopolymerized sol-gel material.

89. The method of claim 85, wherein the providing comprises providing an inorganic PSG.

90. The method of claim 85, wherein the providing comprises providing an inorganic-organic PSG.

91. The method of claim 85, wherein the providing comprises providing a PSG prepared from a metalorganic material.

92. The method of claim 91, wherein the metalorganic material is a metal alkoxide.

93. The method of claim 85, wherein the providing comprises providing a PSG prepared from a photoactive material selected from a metalorganic material that comprises a photoactive group, an organic material that comprises a photoactive group, a photoinitiator, and any combination thereof.

94. The method of claim 85, wherein the providing comprises providing a PSG prepared from a porogen material.

95. The method of claim 85, wherein the providing comprising controlling porosity, pore size, and/or pore shape of the PSG.

96. The method of claim 85, wherein the providing comprises providing a PSG having at least one pore having a diameter in a range of about 0.5 microns to about 20 microns.

97. The method of claim 85, wherein the linking comprises derivatizing the surface of the PSG with a linker.

98. The method of claim 97, wherein the linker comprises a functional group that facilitates the linking of the linker to the surface of the PSG.

99. The method of claim 98, wherein the functional group is an alkoxy group.

100. The method of claim 97, wherein the linker comprises a functional group that facilitates the linking of the enzyme to the linker.

101. The method of claim 100, wherein the functional group is selected from an aldehyde functional group and a succinimide functional group.

102. The method of claim 97, wherein the linker comprises a silicate material.

103. The method of claim 97, wherein the linker comprises a trialkoxysilane.

104. The method of claim 97, wherein the derivatizing is selected from allowing a solution comprising the linker to flow relative to the PSG, pumping a solution comprising the linker relative to the PSG, placing the PSG in a solution comprising the linker, and any combination thereof.

105. The method of claim 97, wherein the derivatizing is for a time of from about 30 minutes to about 60 minutes.

106. The method of claim 97, wherein the derivatizing is at a temperature of from about 4° C. to about room temperature.

107. The method of claim 97, wherein the linking comprises exposing the linker to the enzyme after said derivatizing.

108. The method of claim 107, wherein the exposing is selected from allowing a solution comprising the enzyme to flow relative to the PSG, pumping a solution comprising the enzyme relative to the PSG, placing the PSG in a solution comprising the enzyme, and any combination thereof.

109. The method of claim 107, wherein the exposing is for a time of from about 30 minutes to about 24 hours.

110. The method of claim 107, wherein the exposing is at a temperature of from about 4° C. to a higher temperature sufficient to avoid diminishment of the activity of the enzyme.

111. The method of claim 107, wherein the exposing is at a temperature of from about 4° C. to about 40° C.

112. The method of claim 85, wherein the enzyme comprises an amine functional group.

113. The method of claim 85, wherein the enzyme is selected from trypsin, pepsin, chymotrypsin, malate dehydrogenase, citrate lyase, isocitrate dehydrogenase, and lactate dehydrogenase.

114. The method of claim 85, wherein the linking comprises linking different enzymes on the surface of the PSG.

115. The method of claim 85, wherein the linking is such that the enzyme is covalently linked to a surface in at least one pore of the PSG.

116. The method of claim 85, wherein an activity of the enzyme is enhanced when linked to the surface of the PSG relative to an activity of that enzyme when free of the surface of the PSG.

117. The method of claim 116, wherein the activity is enhanced up to about 200-fold.

118. A method comprising:
providing a photopolymerized sol-gel material (PSG);
linking an enzyme to a surface of the PSG via covalent linkage; and
after said linking, introducing a solution comprising a substrate to the surface of the PSG.

119. The method of claim 118, wherein the providing comprises providing a PSG that comprises a photopolymerized sol-gel material.

120. The method of claim 118, wherein the introducing is facilitated by pressure or voltage.

121. The method of claim 120, wherein the pressure is from about 0.5 to about 20 psi.

122. The method of claim 120, wherein the voltage is from about 1 kV to about 5 kV.

123. The method of claim 118, wherein the introducing is sufficient for the enzyme to at least partially digest the substrate.

124. The method of claim 118, wherein the introducing is for a time from about 5 seconds to that sufficient for at least partial digestion of the substrate.

125. The method of claim 118, wherein the introducing is for a time from about 5 seconds to about 60 minutes.

126. The method of claim 118, wherein the introducing is at a temperature from about 15° C. to about 40° C.

127. The method of claim 118, wherein the substrate comprises a material having a diameter of about a nanometer or more.

128. The method of claim 118, wherein the substrate comprises a component of a biological material.

129. The method of claim 118, wherein the substrate is selected from a biomolecule, a protein, an oligonucleotide, a peptide, a steroid, an organic acid, and any combination thereof.

130. A method comprising:
providing a polymerized sol-gel material (PSG);
linking an enzyme to a surface of the PSG via covalent linkage;
after said linking, introducing a solution comprising a substrate to the surface of the PSG; and
after said introducing, separating any remaining substrate and any product of said introducing from the solution.

131. The method of claim 130, wherein the providing comprises providing a PSG that comprises a photopolymerized sol-gel material.

132. The method of claim 130, wherein the separating is facilitated by pressure or voltage.

133. The method of claim 132, wherein the voltage is from about 1 kV to about 30 kV.

134. The method of claim 130, wherein the separating is selected from chromatographic separation, electrophoretic separation, and any combination thereof.

135. The method of claim 130, further comprising, after said separating, detecting for any remaining substrate and any product of said introducing.

136. The method of claim 135, wherein the detecting is selected from absorption detection, spectroscopic detection, and any combination thereof.

137. A device comprising:
a polymerized sol-gel material (PSG); and
an enzyme covalently linked to a surface of the PSG.

138. The device of claim 137, wherein the PSG is disposed in a sub-device selected from a microreactor, a column, a pipet, a well, and a well plate.

139. The device of claim 138, wherein the sub-device is sufficient to separate an analyte from a sample.

140. The device of claim 137, wherein the PSG comprises a photopolymerized sol-gel material.

141. The device of claim 137, wherein the PSG comprises an inorganic PSG.

142. The device of claim 137, wherein the PSG comprises an organic-inorganic PSG.

143. The device of claim 137, wherein the PSG comprises a PSG prepared from a metalorganic material.

144. The device of claim 143, wherein the metalorganic material is a metal alkoxide.

145. The device of claim 137, wherein the PSG comprises a PSG prepared from a photoactive material selected from a metalorganic material that comprises a photoactive group, an organic material that comprises a photoactive group, a photoinitiator, and any combination thereof.

146. The device of claim 137, wherein the PSG comprises a PSG prepared from a porogen material.

147. The device of claim 137, wherein PSG is of controlled porosity, pore size, and/or pore shape.

148. The device of claim 137, wherein the PSG comprises a PSG having at least one pore having a diameter in a range of about 0.5 microns to about 20 microns.

149. The device of claim 137, wherein the surface of the PSG is derivatized with a linker.

150. The device of claim 149, wherein the linker comprises a functional group that facilitates the linking of the linker to the surface of the PSG.

151. The device of claim 150, wherein the functional group is an alkoxy group.

152. The device of claim 149, wherein the linker comprises a functional group that facilitates the linking of the enzyme to the linker.

153. The device of claim 152, wherein the functional group is selected from an aldehyde functional group and a succinimide functional group.

154. The device of claim 149, wherein the linker comprises a silicate material.

155. The device of claim 149, wherein the linker comprises a trialkoxysilane.

156. The device of claim 149, wherein the enzyme is linked to the surface of the PSG via the linker.

157. The device of claim 137, wherein the enzyme comprises an amine functional group.

158. The device of claim 137, wherein the enzyme is selected from trypsin, pepsin, chymotrypsin, malate dehydrogenase, citrate lyase, isocitrate dehydrogenase, and lactate dehydrogenase.

159. The device of claim 137, wherein different enzymes are linked to the surface of the PSG.

160. The device of claim 137, wherein the linking is such that the enzyme is covalently linked to a surface in at least one pore of the PSG.

161. The device of claim 137, wherein an activity of the enzyme is enhanced when linked to the surface of the PSG relative to an activity of that enzyme free of the surface of the PSG.

162. The device of claim 161, wherein the activity is enhanced up to about 200-fold.

163. The device of claim 137, wherein the enzyme is sufficient to at least partially digest a substrate.

164. The device of claim 163, wherein the substrate comprises a material having a diameter of about a nanometer or more.

165. The device of claim 163, wherein the substrate comprises a component of a biological material.

166. The device of claim 163, wherein the substrate is selected from a biomolecule, a protein, an oligonucleotide, a peptide, a steroid, an organic acid, and any combination thereof.

167. The device of claim 163, further comprising a separation sub-device downstream of the linked-enzyme PSG, the separation sub-device sufficient to separate any remaining substrate and at least one product from the linked-enzyme PSG.

168. The device of claim 167, wherein the separation sub-device is sufficient to separate via any one of chromatographic separation, electrophoretic separation, and any combination thereof.

169. The device of claim 167, further comprising a detection sub-device downstream of the separation sub-device, the detection sub-device sufficient to detect any remaining substrate and at least one product from the separation sub-device.

170. The device of claim 169, wherein the detection sub-device is sufficient to detect via any one of absorption detection, spectroscopic detection, and any combination thereof.
